(19) United States
(12) Patent Application Publication
(10) Pub. No.: US 2008/0207738 A1
(43) Pub. Date: Aug. 28, 2008

(54) DRUG COMBINATIONS TO TREAT DRUG RESISTANT TUMORS

(75) Inventor: Zoltan Kiss, Austin, MN (US)

Correspondence Address: FAEGRE & BENSON LLP;PATENT DOCKETING, 2200 WELLS FARGO CENTER, 90 SOUTH SEVENTH STREET, MINNEAPOLIS, MN, 55402-3901, US

(73) Assignee: CANCURE LABORATORIES, LLC, Austin, MN (US)

(21) Appl. No.: 11/680,142

(22) Filed: Feb. 28, 2007

(51) Int. Cl. A61K 31/35; A61K 31/382; C07D 311/82

(52) U.S. Cl. 514/437; 514/454; 549/388

(57) ABSTRACT

Embodiments of the invention provide a method and composition for treating or reducing multiple drug resistance in cancers. Embodiments of the invention also provide for a xanthene compound to inhibit multidrug resistance protein 1.

### FIELD OF THE INVENTION

The present invention relates to treating drug resistance in cancers. More specifically, the invention relates to treating drug resistance by using a xanthene compound to inhibit multidrug resistant protein 1 (MDR1).

### BACKGROUND

Development of resistance to anti-cancer drugs is a major problem in cancer treatment, be it for solid cancers or cancers of the lymphatic system. Many different mechanisms can contribute to drug resistance that develops during treatment with an anti-cancer drug. Generally, treatment with one anti-cancer drug leads to acquired resistance against several other drugs as well; this phenomenon is called multidrug resistance or MDR. Often, MDR results from increased expression of one or more ATP-dependent efflux pump proteins belonging to the family of 48 known ATP-binding cassette (ABC) transporters subdivided into seven distinct subfamilies (ABA-ABCG) [Gottesman, M. M., Fojo, T. and Bates, S. E. (2001), “Multidrug resistance in cancer: Role of ATP-dependent transporters,” Nature Reviews, 2, 48-58]. Other forms of drug resistance may develop via the increased ability of cancer cells to metabolize anti-cancer drugs, repair drug-induced DNA damage, neutralize the actions of drug-induced, oxygen-derived free radicals, or prevent drug-induced apoptotic cell death.

### SUMMARY OF THE INVENTION

One embodiment of the invention is directed to a method for treating or reducing drug resistance in cancers comprising administering to a patient with cancer a composition comprising a xanthene compound alone or in combination with an anti-cancer agent, a glutathione-reducing agent or both. In some embodiments of the invention, the xanthene compound is 9H-xanthene-9-carboxylic acid-3-{4[2-(4-trimethylsilanyl-methoxy-benzoyloxy)-ethyl]piperazin-1-yl-propyl ester dihydrochloride, or CCcompound104. Another embodiment of the invention is directed towards a composition for treating or reducing drug resistance in cancers comprising administering to a patient with cancer a composition comprising a xanthene compound alone or in combination with an anti-cancer agent or a glutathione-reducing agent or both. Still other embodiments of the invention are directed toward a compound of the formula:

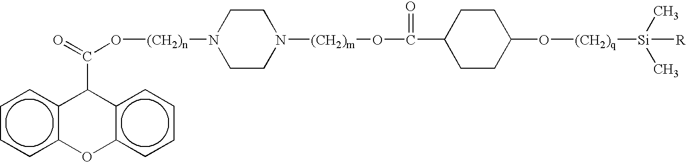

### DETAILED DESCRIPTION OF THE INVENTION

Embodiments of this invention include the use of 9H-xanthene-9-carboxylic acid-3-{4[2-(4-trimethylsilanyl-methoxy-benzoyloxy)-ethyl]-piperazin-1-yl}-propyl ester dihydrochloride (CCcompound104), an inhibitor of MDR1 protein, alone or in combination with other anti-cancer agents to decrease the viability of lymphoid neoplasms or drug resistant cancer cells in solid tumors.

As the first step after identifying the patient with a lymphoid neoplasm or drug resistant solid cancer, tissue and/or blood samples are taken followed by the enrichment of tumor cells. Next, a test is performed to determine the effects of CCcompound104 alone and in combination with other anti-cancer agents on viability of the enriched tumor cells. The best combination is then used for the treatment of cancer patient.

In one embodiment, the viability test is performed with CCcompound104 alone.

In another embodiment, the viability test is performed with CCcompound104 in combination with N,N-diethyl-N-allyl-3-(2-methyl-9H-thioxanthen-9-ylidene)-propan-1-aminium bromide (CCcompound26) or another thioxanthene or thioxanthone compound (for a list, see Table 1).

In a further embodiment, the viability test is performed with CCcompound104 in the presence of ethacrynic acid or another glutathione-depleting agent.

In an additional embodiment, CCcompound104 and the glutathione-depleting agent are used together with a thioxanthene or thioxanthone compound to perform the viability test.

In some embodiments, the viability test with CCcompound104 is performed in the presence of an anti-cancer agent known to serve as a substrate for MDR1, MRP1, (multidrug resistance-associated protein 1) or related drug transporters. For example, the anti-cancer agent can be an anthracycline (epirubicin, doxorubicin), a vinca alkaloid (vincristin, vinblastin), a taxane (paclitaxel, docetaxel), an anthracene (bisantrene, mitoxantrone), an epipodophyllotoxin (etoposide, teniposide), a camphothecin (topocetan, irinotecan/SN-38), or a heavy metal oxyanion (arsenite, trivalent antimony).

In yet other embodiments, CCcompound104 and one or more of the above anti-cancer agents are used together with a glutathione-depleting agent such as ethacrynic acid to perform the viability test.

CCcompound104, CCcompound26 and ethacrynic acid can all be given orally (tablets, gel capsules and other suitable forms) once daily or as frequently as required or needed or as allowed by their toxicity profile. However, they may also be applied by one of the available injection methods (intravenous, subcutaneous, intraarterial, intradermal, intraperitoneal, intratissue). The established anti-cancer agents are administered according to the clinical practice for the given agent and as outlined by relating studies determining the interactions among the combined effects of CCcompound104, ethacryic acid and the anti-cancer agent(s).

Lymphoma is the cancer of the lymphatic system particularly of the abnormally growing lymphocytes. The two major types of lymphoma are Hodgkin's disease and non-Hodgkin lymphoma. Hodgkin disease is a relatively simple disease involving only four main types. In contrast, non-Hodgkin lymphoma (NHL) is a term applied to many different types of lymphatic cancer including the following subtypes; precursor B cell lymphoma, small lymphocytic lymphoma/chronic lymphocytic leukemia, marginal zone lymphomas (nodal marginal zone lymphoma, extranodal MALT, splenic), hairy cell leukemia, follicular lymphoma, mantle cell lymphoma, diffuse large B cell lymphoma, Burkitt's lymphoma, anaplastic large cell lymphoma, peripheral T cell lymphoma and mycosis fungoides. Other lymphoid neoplasms that are not strictly related to non-Hodgkin lymphoma but are covered by this invention includes acute lymphoblastic leukemia, lymphoplasmacytoid lymphoma, T-cell chronic lymphocytic leukemia/prolymphocytic leukemia, and any other cancers of lymphoid origin that are not easily classified. The commonly accepted term “lymphoid neoplasm” as used here includes all cancers listed above; they are all within the scope of the invention.

The Active Components.

### 9H-xanthene-9-carboxylic acid-3-{4[2-(4-trimethylsilanyl-methoxy-benzoyloxy)-ethyl]piperazin-1-yl-propyl ester dihydrochloride (referenced as CCcompound104).

The general structure of xanthene compounds is presented below in scheme 1.

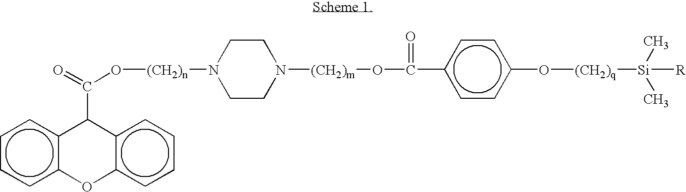

In this general structure: n=1-4
a. m=1-4
b. q=1-3
c. R=methyl, ethyl, propyl, butyl, or phenyl
d. Si may be replaced with N

The position of the side chain may be ortho or para.

As used in this application, MDR1 refers to β-glycoprotein that is an ABC transporter. In an MDR1 overexpressing lymphoma cell line, CCcompound104 strongly enhanced the accumulation Rhodamine 123, a substrate of MDR1. Prevention of efflux of rhodamine 123 suggests that CCcompound104 is an effective inhibitor of MDR1 activity. Inhibition of MDR1 activity allows retention of anti-cancer compounds that are substrates of this drug transporter; anti-cancer compounds, for example, include anthracycline (epirubicin, doxorubicin), a vinca alkaloid (vincristin, vinblastin), a taxane (paclitaxel, docetaxel), an anthracene (bisantrene, mitoxantrone), an epipodophyllotoxin (etoposide, teniposide), a camphothecin (topocetan, irinotecan/SN-38), or a heavy metal oxyanion (arsenite, trivalent antimony). Embodiments of the invention demonstrate that in the MDR1 overexpressing lymphoma cell line CCcompound104 enhances the anti-proliferative effects of epirubicin. Thus, in MDR1 expressing tumors CCcompound104 may be used to enhance accumulation of MDR1 substrate anti-cancer drugs and thereby enhance their anti-cancer effects.

In one embodiment, CCcompound104 is used to enhance the anti-proliferative effects of CCcompound26 in various lymphoma and lymphocytic leukemia cells as well as cancer cells derived from solid tumors.

In other embodiments, CCcompound104 is used to kill lymphocytic leukemia cells.

In other embodiments, CCcompound104 is used to inhibit MDR1 activity.

CCcompound26 and related compounds.

The synthesis of CCcompound26 [N,N-diethyl-N-allyl-3-(2-methyl-9H-thioxanthen-9-ylidene)-propan-1-aminium bromide] and several related thioxanthene and thioxanthone compounds and their selenium containing derivatives listed below in Table 1 has been disclosed in a recent U.S. patent application (filed on Jul. 19, 2006; application Ser. No. 11/458,502, entitled “Compounds and compositions to control abnormal cell growth,”; inventor, Zoltan Kiss), which application is incorporated herein by reference. In some embodiments, CCcompound26 was described as an inhibitor of proliferation of various types of cancer cells compared to normal cells. In embodiments of the present invention, CCcompound26 is used together with CCcompound104 to decrease the viability of lymphoma and lymphocytic leukemia cells as well as cancer cells derived from solid tumors that are known or suspected to express MDR1. At the relatively low concentrations used (1-5 μM) CCcompound26 alone usually has little or no effect on the viability of cancer cells; it exerts significant inhibitory effects in the presence of CCcompound104.

TABLE 1
A Representative List of Thioxanthone and Thioxanthene CC
compounds and their Selenium-Containing Derivatives Used in Embodiments
of the Invention.

| Trivial name | Chemical name | Structure |
|---|---|---|
| CCcompound1 | [3-(3,4-Dimethyl-9-oxo-9H-thioxanthen-2-yloxy)-2-hydroxypropyl]trimethyl-ammonium chloride | 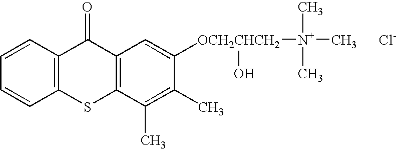 |
| CCcompound2 | N,N,N-Trimethyl-2-[(9-oxo-9H-thioxanthen-2-yl)methoxy]-ethanaminiumiodide | 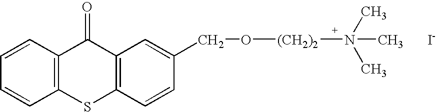 |
| CCcompound3 | N,N-Diethyl-N-methyl-2-[9-oxo-9H-CCDTHTthioxanthen-2-yl)methoxy]-ethanaminium iodide | 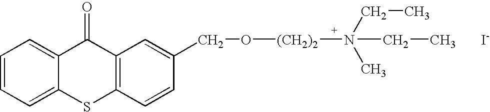 |
| CCcompound4 | N,N,N-Triethyl-2-[(9-oxo-9H-thioxanthen-2-yl)methoxy]-ethanaminiumiodide | 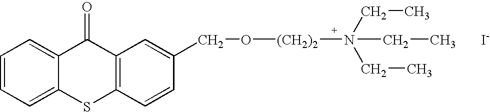 |
| CCcompound5 | N-Ethyl-N,N-dimethyl-2-[(9-oxo-9H-thioxanthen-2-yl)methoxy]-ethanaminiumiodide | 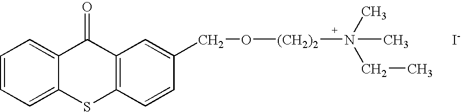 |
| CCcompound6 | 2-{[2-(Diethylamino)ethoxy]methyl}-9H-thioxanthen-9-onehydrochloride | 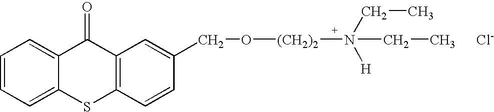 |
| CCcompound7 | N,N,N-Trimethyl-2-[(9-oxo-9H-thioxanthen-2-yl)methoxy]-propan-1-aminium iodide | 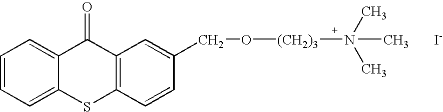 |
| CCcompound8 | 2-{[2-(Diethylamino)propoxy]methyl}-9H-thioxanthen-9-onehydrochloride | 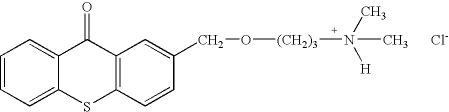 |
| CCcompound 9 | N,N,N-Triethyl-3-[(9-oxo-9H-thioxanthen-2-yl)methoxy]-propan-1-aminium iodide | 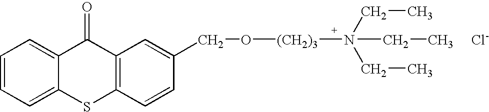 |
| CCcompound10 | N,N-Diethyl-N-methyl-3-[(9-oxo-9H-thioxanthen-2-yl)methoxy]-propan-1-aminium iodide | 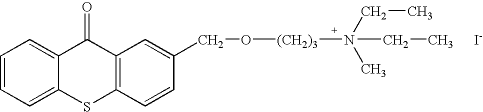 |
| CCcompound11 | N,N-Dimethyl-N-ethyl-3-[(9-oxo-9H-thioxanthen-2-yl)methoxy]-propan-1-aminium iodide | 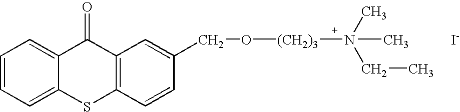 |
| CCcompound12 | 2-{[3-(Diethylamino)propoxy]methyl}-9H-thioxanthen-9-onehydrochloride | 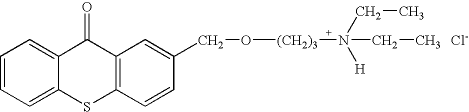 |
| CCcompound13 | 2-Hydroxy-N,N-dimethyl-N-[(9-oxo-9H-thioxanthen-2-yl)methyl]-ethanaminiumbromide | 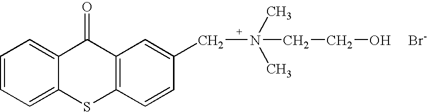 |
| CCcompound14 | 2-Hydroxy-N,N-Diethyl-N-[(9-oxo-9H-thioxanthen-2-yl)methyl]-ethanaminiumbromide | 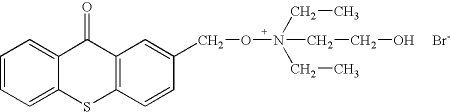 |
| CCcompound15 | 3-Hydroxy-N,N-dimethyl-N-[(9-oxo-9H-thioxanthen-2-yl)methyl]propan-1-aminium bromide | 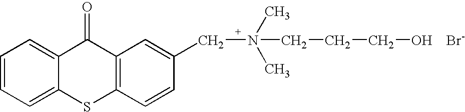 |
| CCcompound16 | 3-Hydroxy-N,N-diethyl-N-[(9-oxo-9H-thioxanthen-2-yl)methyl]-propan-1-aminium bromide | 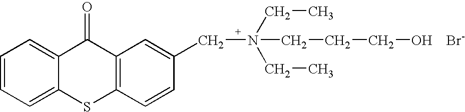 |
| CCcompound17 | 3-(9-hydroxy-9H-thioxanthen-9-yl)-N,N,N-trimethyl-propan-1-aminiumiodide | 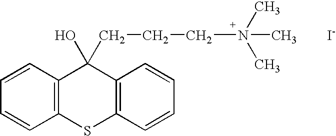 |
| CCcompound18 | 3-(9-hydroxy-9H-selenoxanthen-9-yl)-N,N,N-trimethyl-propan-1-aminiumiodide | 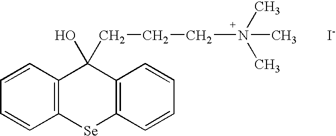 |
| CCcompound19 | N,N,N-trimethyl-3-(9H-thioxanthen-9-ylidene)-propan-1-aminium iodide | 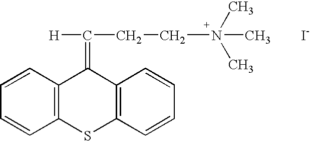 |
| CCcompound20 | N,N,N-trimethyl-3-(9H-selenoxanthen-9-ylidene)-propan-1-aminium iodide | 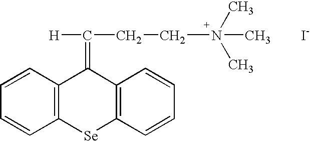 |
| CCcompound21 | N,N,N-trimethyl-3-(2-methyl-9H-thioxanthen-9-ylidene)-propan-1-aminiumiodide | 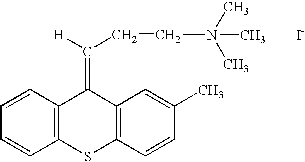 |
| CCcompound22 | N,N-Dimethyl-N-ethyl-3-(2-methyl-9H-thioxanthen-9-ylidene)-propan-1-aminiumiodide | 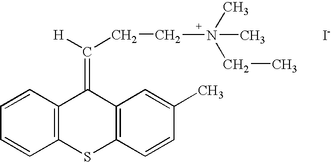 |
| CCcompound23 | N,N-Diethyl-N-methyl-3-(2-methyl-9H-thioxanthen-9-ylidene)-propan-1-aminiumiodide | 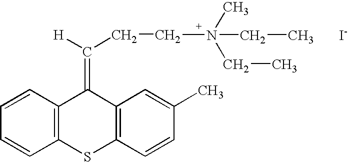 |
| CCcompound24 | N,N-Dimethyl-N-allyl-3-(2-methyl-9H-thioxanthen-9-ylidene)-propan-1-aminiumbromide | 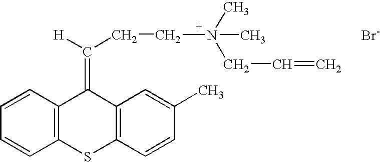 |
| CCcompound25 | N,N,N-triethyl-3-(2-methyl-9H-thioxanthen-9-ylidene)-propan-1-aminium iodide | 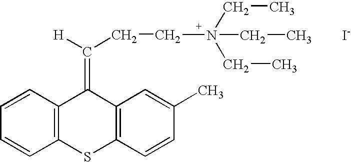 |
| CCcompound26 | N,N-Diethyl-N-allyl-3-(2-methyl-9H-thioxanthen-9-ylidene)-propan-1-aminiumbromide | 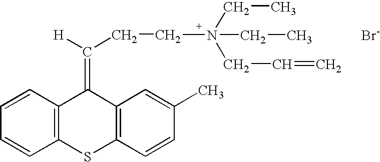 |

Ethacrynic acid and other glutathione-depleting agents.

Transport of anti-cancer agents by a multidrug-resistance associated protein 1 (MRP1), depends on the co-transport of glutathione. An agent, like ethacrynic acid (EA) that can reduce the cellular content of glutathione is expected to inhibit the activity of MRP1. Conversely, if EA can enhance the effect of an anti-cancer agent, this suggests that the anti-cancer agent is extruded from the cytosol by MRP1 and that the action of EA is due to the inhibition of MRP1.

In one embodiment of the invention, EA and CCcompound104 synergistically reduce cell viability in MRP1-expressing lymphoma and leukemia cells as well as breast cancer cells. In other embodiments, another glutathione-reducing agent may be used in the treatment of lymphoma and lymphocytic leukemia. For example, other glutathione-reducing agents include piriprost, buthionine sulfoximide, 1-chloro-2,4-dinitrobenzene, or chlorambucil (all available commercially).

Methods of Use

In one embodiment, CCcompound104, CCcompound26 and EA may be administered orally. The two CC compounds are water-soluble, while EA is first dissolved in absolute ethanol and diluted in water or a suitable physiologically compatible liquid solvent. In one embodiment of the invention, the CC compounds and EA are in the form of a tablet, gel capsule, a liquid, or the like. In each case, the compounds are mixed with one or more carriers chosen by one having ordinary skill in the art to best suit the goal of treatment. In addition to the active compounds, the tablet or gel may contain any component that is presently used in the pharmaceutical field to ensure firmness, stability, solubility and appropriate taste. Any additional component of the tablet or gel will be chemically inert; i.e., it will not participate in a chemical reaction with the active compounds or the other additives. Each compound may be prepared for oral consumption separately, or the tablet, gel capsule or liquid may contain any of the two components or all three components. Tablets containing 25 mg or 50 mg of EA can be purchased from Merck.

In another embodiment, the selected compound or compounds are mixed in a bio-compatible liquid carrier, such as physiological saline (0.9% NaCl), and injected via one of the systemic routes such as, for example, intravenous, intraarterial, intraperitoneal, subcutaneous, intraportal, intracranial or intradermal. In other embodiments, the CC compound/EA-containing solution is directly injected into the affected lymphatic organ or bone marrow or solid tumor. The CC compound/EA-containing solution may also be administered via infusion or a subcutaneously implanted minipump over a prolonged period of time in a controlled fashion.

Various combinations of the application methods may be used. For example, application of one or both CC compound(s) via injection or infusion may be used. Injection or infusion administration of CC compound(s) then may be combined with an oral administration of the glutathione-reducing drug.

The therapeutic amount of the selected compound(s) is determined by the application method, the nature of the target (solid tumor, lymphatic organs/lymph nodes or bone marrow and/or leukemia cells present in the circulation), the stage of the disease, combination with other treatments, and the age of the patient. The health care provider who possesses all the required information may determine the required therapeutic amount. For example, in the case of oral administration, CC compounds may be applied at doses between 10-1,000 mg per m² body surface. EA may be applied orally at doses ranging between 25-75 mg per m² body surface [O'Dwyer, P. J., LaCreta, F., Nash, S., Tinsley, P. W., Schilde, R., Clapper, M. I., Tew, K. D., Panting. l., Litwin, S., Comis R. L. and Ozols, R. F. (1991), “Phase I study of thiotepa in combination with glutathione transferase inhibitor ethacrynic acid,” Cancer Res., 51, 6059-6065]. In embodiments where a dose is administered by infusion or intravenous, subcutaneous or intraperitoneal injection, one dose may contain between about 5 to 500 mg of CC compound per m² body surface. In other embodiments, where a dose is administered by either infusion or an injection method, EA may be administered at 5-25 mg per m² body surface.

The frequency of administrations of CC compounds and the glutathione-reducing agent is dependent on whether CCcompound104 will be used alone or together with CCcompound26 and/or the glutathione-depleting agent.

In some embodiments, chlorambucil, which is not only a glutathione-reducing but also an alkylating agent employed in the treatment of several neoplastic diseases including chronic lymphocytic leukemia (CLL), may be used. The less tolerated the CC compounds are the less amount of chlorambucil may be given together with CC compounds. In some embodiments, chlorambucil may be given orally at 12 mg per m² body surface dose daily for 7 consecutive days followed by repeating such course several times, always including a 28-day chlorambucil-free period between courses. EA may also be replaced with other glutathione-depleting agents as listed above.

The CC compound/EA-containing compositions may include various additions including other anti-cancer agents or protective agents. The criterion for using another agent is that it increases, or at least does not decrease, the effectiveness of the active components in achieving the desired beneficial effect. An incomplete list of anti-cancer compounds that may be used in combination with a CC compound/EA-containing composition includes purine analogs (fludarabine, clofarabine, cladribine, nelarabine, 2-chlorodeoxyadenosine), corticosteroids such as prednisone, cytotoxic agents (cyclophosphamide, adriamycine, and vincristine), monoclonal antibodies (rituximab, alemtuzumab), radiolabeled monoclonal antibodies (ibritumomab, tiuxetan, tositumomab), etoposid, cisplatin or carboplatin, and various combinations of these agents with each other.

The CC compound/EA-containing compositions may also include one or more protective agents that reduce or prevent cachexia or other toxic side effects of chemotherapy. Amifostine is such a protective agent. Other protective agents that may be used include a series of CC compounds such as CCcompound1 and CCcompound3 (also listed in Table 1 as potential anti-cancer agents) [U.S. patent application Ser. No. 11/558,533, filed on Oct. 10, 2006, and entitled “Thioxanthone compounds to reverse weight loss,” to Zoltan Kiss], and incorporated in its entirety herein by reference, and placental alkaline phosphatase [U.S. patent application Ser. No. 11/463,022, filed on Aug. 8, 2006, and entitled “Use of placental alkaline phosphatase to maintain healthy tissue mass in mammals,” to Zoltan Kiss], incorporated herein by reference.

Autologous or allogeneic hematopoietic stem cell transplantation accompanied by the administration of promoters of survival and proliferation of hematopoietic stem cells and progenitors may also be used to aid CC compound/EA-based therapy. Examples of promoters include colony-stimulating factor and granulocyte colony-stimulating factor. Other examples include the combination of placental alkaline phosphatase and transferrin [U.S. patent application Ser. No. 11/560,167, filed on Nov. 15, 2006, and entitled “Combinations of human proteins to enhance viability of stem cells and progenitor cells,” to Zoltan Kiss], incorporated herein by reference. In this procedure, CC compounds and EA may be used prior to and after the transplantation event.

The CC compound/EA-containing compositions can be stored at room temperature for at least one year and at 40° C. for several years under aseptic conditions.

The CC compound-containing compositions can be made using a number of suitable techniques. In some embodiments, the CC compound and a carrier are mixed together within a commercial mixer to form a solution, a suspension, or the like. In pharmaceutical composition embodiments, methodologies for the formulation and preparation of tablets, gels, and liquids are well known, and can be found, for example, in Remington's Pharmaceutical Sciences, Eighteenth Edition, A. R. Gennaro, Ed., Mack Publishing Co. Easton, Pa. 1990, incorporated hereby by reference.

CC compound/EA-containing compositions may be used in combination or alternatively with anti-proliferation treatments other than chemotherapy, such as radiation and surgery, for example. In one embodiment, the CC compound/EA-containing solution is applied by an injection method after completing a course of radiotherapy to prevent recurrence of the primary tumor or development of secondary tumors.

As can be seen, various combinations of drugs may be used. Examples include CCcompound104 alone, CCcompound104+CCcompound26, CCcompound104+ethacrynic acid, CCcompound104+chlorambucil, CCcompound104+CCcompound26+ethacrynic acid, CCcompound104+CCcompound26+chlorambucil, CCcompound104+CCcompound26+ethacrynic acid+another anti-cancer agent, CCcompound104+CCcompound26+chlorambucil+another agent (selected from the list provided above).

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof. All patents, patent applications and publications are hereby incorporated by reference.

### EXAMPLES

### Example 1

### Synthesis of CCcompound104 or 9H-xanthene-9-carboxylic acid-3-{4[2-(4trimethylsilanyl-methoxy-benzoyloxy)-ethyl]piperazin-1-yl-propyl ester dihydrochloride

Structure:

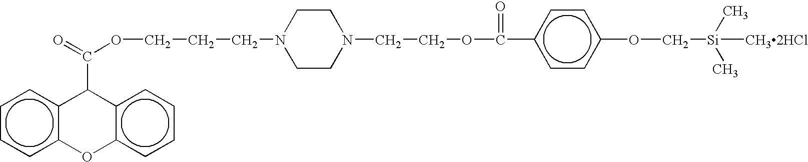

### Step 1: Synthesis of 4-trimethyl silylmethyl methyl benzoate (C₁₂H₁₈O₃Si)

Structure:

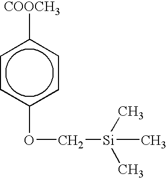

NaH (4.4-g), supplied as 60% oily suspension, was washed over a glass filter with 2×50-ml absolute dimethylformamide to remove oil. In small portions, NaH was added slowly (over a 20 min time period) to 16.8-g of 4-hydroxymethylbenzoate previously dissolved in dimethylformamide (at room temperature with constant mixing with magnetic stirrer); mixing continued for 30 min after the last addition. The resulting suspension was placed on a 60° C. oil bath followed by drop-wise addition of 16.7-g of trimethylbromomethyl silane, previously dissolved in 50-ml dimethylformamide. The mixture was mixed for another 1-hour period and then taken off the oil bath. After the mixture reached the room temperature, the surplus NaH was reacted with 30-ml methanol to form NaOH and H₂. The precipitated NaOH was removed by filtering on a glass filter and the filtrate was subjected to distillation (2-3 Hgmm). The resulting brown precipitate was dissolved in 200-ml of ethylacetate followed by washing with 2×150 ml distilled water. The organic phase was dried over water-free sodium sulfate followed by the removal of ethylacetate by distillation using a Rotadest equipment. The resulting light yellow oily substance (4-trimethyl silylmethyl methylbenzoate) was used for the next step as the starting material. The yield of this product was 82%.

### Step 2: Synthesis of 4-trimethylsilylmethyl benzoic acid (C₁₁H₁₆O₃Si)

Structure:

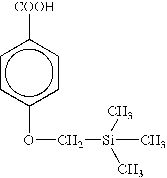

4-trimethyl silylmethyl methylbenzoate (23.83-g) was dissolved in 120-ml of methanol followed by the addition of 20-ml of 8N of NaOH. The mixture was mixed at room temperature for 26-28 hours. Then, the mixture was poured onto 50-g crushed ice followed by adjustment of pH to 1.0 with 10N of HCl. The precipitated white crystals were filtered and washed over the filter with 2×100-ml of ice-cold water. The product was dried under infrared light; the yield was ˜86%.

### Step 3: Synthesis of 4-trimethyl silylmethyl benzoic acid chloride (C₁₁H₁₅ClO₂Si)

Structure:

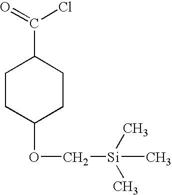

4-trimethyl silylmethyl benzoic acid was suspended in 80-ml absolute chloroform (CHCl₃) followed by drop-wise addition (over a 10-min period) of 9.2-ml of thionylchloride (SOCl₂) during constant mixing (using magnetic stirrer). The mixture was refluxed under N₂ for 8 hours. The solvent and surplus SOCl₂ was removed by distillation (Rotadest). The remaining material is rapidly crystallized at 4° C. The yield of this product was nearly 100%.

### Step 4: Synthesis of 3-chloropropyl-9H-xanthene-carboxylate (C₁₇H₁₄CIO₃)

Structure:

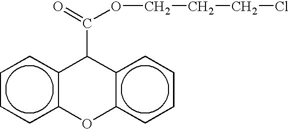

9-Xanthene carboxylic acid (20-g) was dissolved in 90-ml absolute dioxane. This was followed by the addition of 15.83-g of 1-Cl-3-propanol dissolved in 20-ml dioxane and then by the drop-wise (over about 10 min period) addition of 14-ml of sulfuric acid (H₂SO₄) at room temperature during continuous stirring. The mixture was kept in an oil bath (120-125° C.) under gentle reflux for 24 hours. After cooling down to room temperature, the mixture was poured into 300-ml ice-cold distilled water followed by extraction with 3×100-ml of diethylether. The combined diethylether phase was sequentially washed with 2×100-ml distilled water, 4×150-ml of 3% ice-cold sodium carbonate (Na₂CO₃) solution, and 150-ml of saturated sodium chloride (NaCl) solution. The diethylether phase was dried on water-free sodium sulfate (Na₂SO₄) followed by the removal of solvent by distillation. The resulting dark brown oily material still contained some 1-Cl-3-propanol that was removed under vacuum. The yield was 81%; purity˜99%.

### Step 5: Synthesis of 3-iodopropyl-9H-xanthene carboxylate (C₁₇H₁₄O₃)

Structure:

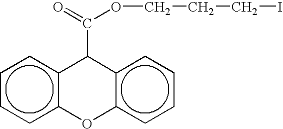

Water-free NaI (20.2-g) was added to 21.7-g of 3-chloropropyl-9H-xanthene-carboxylate previously dissolved in 120-ml of absolute acetone. The mixture was refluxed and stirred for 30 hours leading to continuous precipitation of a white substance (NaCl). The mixture was let to cool to room temperature, then the precipitated NaCl was removed by filtration. After washing the precipitate on the filter three times with 20-ml absolute acetone, the filtrate was subjected to distillation (Rotadest). The oily product was suspended in 250-ml of diethylether and then sequentially washed with 2×80 ml of distilled water, 2×80 ml of 5% NaHSO₃ (sodium hydrogen sulfite), and 80-ml of saturated NaCl solution. The product was dried on water-free sodium sulfate and the remaining solvent was removed by distillation. The product's purity at this stage was 90%. To achieve near 100% purity, the product was suspended in 20-ml of absolute acetone followed by column chromatography on Al₂O₃ (activity III according to Brockman) using 250-ml of hexane to wash the column. After evaporation of the hexane phase the remaining light yellow product was an oily material. The yield of this product was 76%.

### Step 6: Synthesis of 3-[4-(2-hydroxyethyl)-piperazine-1-yl]-propyl-9H-xanthene carboxylate (C₂₃H₂₇N₂O₄)

Structure:

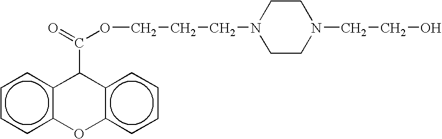

3-Iodopropyl-9H-xanthene carboxylate (15.0-g) was dissolved in 30-ml absolute acetone followed by the addition of 10.4-g of β-hydroxyethylpiperazine during constant mixing at room temperature. After about 35 min a solid material began to precipitate along with an increase of temperature to 40-45° C. (the temperature will return to about 24° C. after 1-2 hours). The mixture was mixed for 44 hours followed by filtration and washing of precipitate 3-times with 20-ml absolute acetone. After distillation of the filtrate, the remaining solid material was dissolved in 110-ml chloroform then washed 5-times with 70-ml distilled water to remove remaining β-hydroxyethylpiperazine. The chloroform phase was extracted 3-times with 70-ml of 1N HCl, then the pH of the acid-water phase was adjusted to 12 with solid sodium carbonate followed by extraction with 3×60-ml of chloroform. The chloroform phase was dried with potassium carbonate (K₂CO₃) followed by distillation of the chloroform phase. The oily-solid product was dissolved in 150-ml of ethylacetate, treated with charcoal, refluxed for 3 min and filtered. The product is a light-yellow oily substance. The yield was 77%.

### Step 7: Synthesis of the Final Product; 9H-xanthene-9-carboxylic acid-3-{4[2-(4-trimethylsilanyl-methoxy-benzoyloxy)-ethyl]piperazin-1-yl-propyl ester dihydrochloride or CCcompound104

3-[4-(2-hydroxyethyl)-piperazine-1-yl]-propyl-9H-xanthene carboxylate (3.16-g) and 4-trimethyl silylmethyl benzoic acid chloride (2.2-g), dissolved in 40-ml and 20-ml absolute acetone respectively, were mixed and continuously stirred for 22.5 hours. After 2.5 hours the solid product began to precipitate. At this point, 25-ml of absolute diethylether was added followed by heating the mixture to about 36° C. (by keeping the mixture in a 50° C. oil bath; this temperature was kept for the rest of incubation). The precipitated crystals were separated by filtration then washed with 2×10-ml of diethylether. The crystals were dissolved in 30-ml of absolute ethanol, cooled to 4-8° C., then its pH was adjusted to 1.0 with a mixture of absolute ethanol and HCl [dried HCl gas is lead to ethanol until the concentration of HCl reaches about 20%]. The mixture was kept at 4° C. for 12 hours. This procedure leads to the precipitation of HCl salt of CCcompound104 which was filtered (glass filter) and washed with 2×10-ml absolute diethylether. The purity and yield of the final product was 100% and 77%, respectively.

### Example 2

### Cell Lines

The human breast cancer MCF-7 and MCF-7/ADR (also called MCF-7/MDR1) cells as well as the murine lymphocytic leukemia P388 and P388/ADR (also called P388/MDR1) cells were obtained from the National Cancer Institute, Bethesda, Md., Developmental Therapeutics Program Tumor Repository. These cell lines are frequently used models for the study of drug resistance [see, for example, Peer, D., Dekel, Y., Malikhov, D. and Margalit, R. (2004), “Fluxetine inhibits multidrug resistance extrusion pumps and enhances responses to chemotherapy in syngeneic and in human xenograft tumor models,” Cancer Res., 64, 7562-7569]. Importantly, the P388/MDR1 cell line that was used contains only about 6-times more MDR1 protein than the parent cell line. This resembles the situation in human tumors that usually express only 2-5-fold more MDR1 than the normal tissue.

The two MCF-7 cell lines were grown in Richter's Iscove's modified Eagle medium (IMEM) supplemented with 2 mM glutamine, 12 mg/ml L-proline, 50 μg/ml of gentamycin and 10% fetal bovine serum. The MCF-7/ADR cells that were selected by incubating MCF-7 cells with increasing concentrations of adriamycin (doxorubicin) were periodically reselected by growing them for 2 weeks in a medium containing 10 μM adriamycin. The two P388 cell lines were grown in Fischer's medium in the presence of 10% horse serum. The P388/ADR cell line also was reselected with adriamycin as described for the MCF-7/ADR cells.

The L5178 (parent) mouse T-cell lymphoma cells and the human MDR1-transfected subline (L5178/MDR1) [Aszalos, A., Pine, P. S., Pandey, R. and Gottesman, M. M. (1995), “Behaviour of N-acetylated daunorubicins in MDR1 gene transfected and parental cells,” Biochem. Pharmacol., 50, 889-892] were donated by the Food and Drug Administration, Division of Research and testing (Washington, D.C.). The cells were grown in McCoy's 5A medium supplemented with 10% heat-inactivated horse serum, L-glutamine and 50 μg/ml of gentamycin. It should be noted that the L5178/MDR1 cells express MDR1 at about 80 to 90-times higher level compared to the parent cell line. Thus, while this cell line is suitable to demonstrate the specific role of MDR1 in drug resistance, probably much higher concentration of MDR1 inhibitor is required to block MDR1 activity than in case of human tumor cells.

### Example 3

### Separation of Lymphocytes

Heparinized peripheral-blood samples were obtained from chronic lymphocytic leukemia patients with more than 70% malignant cells after informed consent, in accordance with the Declaration of Helsinki. The blood samples (10-ml) were diluted 1:1 with cold phosphate buffered saline (PBS; 0.135 M NaCl, 2.7 mM KCl, 1.5 mM KH₂PO₄, 8 mM Na₂HPO₄ [pH 7.4]) and layered onto Ficoll-Hypaque (8-ml, specific gravity, 1.086; Life Technologies, Grand Island, N.Y.). Then, the blood was centrifuged at 433 g for 20 min, and mononuclear cells were removed from the interphase. Cells were washed twice with cold PBS and after each centrifugation step the pelleted cells were resuspended in 10-ml RPMI 1640 supplemented with 10% fetal bovine serum (inactivated for 30 min at 56° C.). A Coulter channelyzer (Coulter Electronics, Hialeah, Fla.) was used to determine the cell number. Then the number of cells was adjusted to 1×107 cells per 1 ml before using them for the experiments.

### Example 4

### Determination of Cell Viability

For the determination of relative number of viable cells before and after treatments, in most cases the MTT assay was used. This calorimetric assay is based on the ability of living cells, but not dead cells, to reduce 3-(4, 5-dimethyl thiazol-2-yl)-2, 5-diphenyltetrazolium bromide [Carmichael, J, De Graff, W. G., Gazdar, A. F., Minna, J. D. and Mitchell, J. B. (1987), “Evaluation oftetrazolium-based semiautomated calorimetric assay: Assessment of chemosensitivity testing,” Cancer Res., 47, 936-942]. For this assay, cells were plated in 96-well plates (10,000-30,000 cells in case of cultured cells, 100,000 cells in case of healthy lymphocytes, and 200,000 cells in case of chronic lymphocytic leukemia or non-Hodgkin lymphocytes) in the respective incubation media. After a 24-h resting period, the cells were treated with the test agents and then incubated (in CO₂ incubator under humidified atmosphere) for 72 hours (final incubation volume, 150 μl). The MTT assay was performed both with the untreated and treated cell cultures at the conclusion of the incubation. Often, the MTT assay also was performed at the start of treatments to allow assessment of proliferation rates in the control and treated cell cultures. All data presented are the average of 4 incubations (in 4 separate wells) with the same cell population. In each case, the difference between the lowest and highest values was less than 15%.

### Example 5

### Analysis of Cell Apoptosis

Cell apoptosis was analyzed in drug-treated L5178/MDR1 cells using flow cytometry. This method for which a Detection Kit I (PharMingen, San Diego, Calif.) was used is suitable to detect cell membrane binding of annexin V that specifically and proportionally increases if cell death occurs by the apoptotic mechanism. Untreated or treated cells were washed with PBS and resuspended in 0.2 ml of 1× annexin binding buffer (BD Biosciences, San Jose, Calif.) at a concentration of 10⁶ cells per 1 ml. Annexin V-FITC (5 μl) was added and the cells were incubated in the dark for 15 minutes at room temperature. Then the labeled cells were added to 10-μl propidium iodide (50 μg/ml) and analyzed immediately with a FACSCALIBUR cytometer (Beckton-Dickinson). Data from at least 10,000 events per sample were recorded and processed using CellQuest software (Becton-Dickinson). The results were obtained after subtracting endogenous level of annexin binding in untreated timed-control samples. Such flow cytometric analysis revealed that after treatment of cells with 5 μg/ml of CCcompound104+25 μM CCcompound26 for 72 hours, which killed 88% of cells (see Table 6 for results), 85-90% of cells died by the apoptotic mechanism. In a similar experiment, combined treatment with 5 μg/ml of CCcompound104+2.5 μg/ml of epirubicin for 72 hours, which killed 100% of cells (see Table 14), also caused about 80-85% of the cells die by the apoptotic cell death pathway. Thus, these drug combinations can be used without triggering major side effects that might be caused by the necrotic form of cell death.

### Example 6

### Determination of Reversing Effect of CCcompound104 on Multidrug Resistance

The L5178 mouse T cell lymphoma parent cell line and its MDR1 expressing subline (L5178/MDR1) were used. The MDR1 transfected cells expressed 80-90-times more MDR1 protein than the parent cell line. The cells were adjusted to a concentration of 2×10⁶ cells per ml, then resuspended in serum-free McCoy's 5A medium and 0.5-ml portions distributed into small (1.2-ml) Eppendorf centrifuge tubes. After adding various concentrations of test compounds as listed below in Table 2, the samples were incubated for 10 min at room temperature (22° C.). Then 10-μl (5.2 mM final concentration) of Rhodamine 123 indicator was added to the cells and incubations continued for 20 min at 37° C. in a water bath. After centrifugation (450 g, 10 min) the pelleted cells were washed twice with PBS and resuspended in 0.5-ml of PBS for cytometry. The fluorescence intensity of the cell population was measured by flow cytometry using Beckton Dickinson's FACScan instrument. Verapamil and cyclosporin were used as positive controls.

The percentage mean fluorescence intensity (FL-1) was calculated for the treated parental and MDR1 cell lines as compared with that of untreated cells. The activity ratio (R) reflects the fold increase of retention of Rhodamine 123 in treated versus untreated MDR1 cells. The higher the “R” value is, the greater the accumulation of Rhodamine 123 in the cells, i.e. the greater the effect of test compound on the reversal of drug resistance. Other values measured are Forward scattered count (FSC) and site scattered count (SSC).

The results from a representative experiment, presented in Table 2, indicate that CCcompound104 is a potent inhibitor of MDR1 activity. In the absence of an MDR1 inhibitor, the MDR1 expressing L5178/MDR1 cells (indicated as “MDR1” in Table 2) retained very little Rhodamine 123 compared to the parent L5178 cells (indicated as “Parent” in Table 2), confirming a high level of MDR1 expression and activity. In the MDR1 cells, at an optimal concentration (4 μg/ml) CCcompound104 enhanced cellular content of Rhodamine 123 about 107-fold (R=107.78). In comparison, optimal concentrations of verapamil (10 μg/ml) and cyclosporin (0.4 μg/ml) enhanced retention of Rhodamine 123 about 4-fold and 17-fold, respectively. The large effects of CCcompound104 were highly reproducible in 4 other experiments in which 4 μg/ml of CCcompound104 enhanced Rhodamine 123 retention 97-fold, 88-fold, 36-fold, and 119-fold. In the same experiments, the effects of verapamil varied between 3-30-fold. In similar experiments with the parent cells, CCcompound104 had no detectable effects on the cellular content of Rhodamine 123 (data not shown). This confirms that CCcompound104 specifically affects Rhodamine 123 uptake via inhibiting MDR1 activity.

TABLE 2
Comparison of effects of CCcompound104, verapamil, and
cyclosporin on the cellular content of Rhodamine 123.

| Treatment | μg/ml | FSC | SSC | FL-1 | R |
|---|---|---|---|---|---|
| Parent, control | — | 511.25 | 199.86 | 980.67 | — |
| MDR1, control | — | 492.28 | 210.37 | 7.33 | 00 |
| MDR1, verapamil | 10 | 471.33 | 211.63 | 28.45 | 3.88 |
| MDR1, cyclosporine | 0.4 | 501.46 | 223.48 | 122.68 | 16.74 |
| CCcompound104 | 4.0 | 494.58 | 209.80 | 790.02 | 107.78 |
| CCcompound104 | 0.4 | 569.45 | 197.44 | 161.84 | 22.07 |
| CCcompound104 | 0.04 | 582.30 | 192.80 | 30.85 | 4.20 |

### Example 7

### Combined Effects of CCcompound104 and CCcompound26 on the Viability of Parent P388 Murine Lymphocytic Leukemia Cells

Parent P388 cells were seeded either at 10,000 cells per well or 30,000 cells per well in 96-well plates and then incubated for 72 hours in the absence or presence of CCcompound104 and CCcompound26 as indicated in Table 3. When cells were seeded at 10,000 cells per well, 2.5 μg/ml of CCcompound104 alone decreased the number of viable cells by 96% (Table 3). In contrast, when cells were seeded at 30,000 cells per well, even 5 μg/ml of CCcompound104 alone decreased the number of viable cells only by 87%. One hundred percent decrease in the number of viable cells required simultaneous treatments with 2.5 μg/ml of CCcompound104 and 1 μM CCcompound26 (Table 3). Overall, the data indicate that P388 lymphocytic leukemia cells not expressing MDR1 are sensitive to the inhibitory action of CCcompound104 but at higher cell numbers efficient (100%) killing of these cells requires the co-presence of CCcompound26.

TABLE 3
Combined effects of CCcompound104 and CCcompound26 on the
viability of parent P388 cells at lower and higher cell numbers.

| 10,000 cells | | | 30,000 cells | | |
|---|---|---|---|---|---|
| CC104* | CC26** | Inhibition | CC104 | CC26 | Inhibition |
| (μg/ml) | (μM) | (%) | (μg/ml) | (μM) | (%) |
| 2.5 | — | 94 | 2.5 | — | 41 |
| — | 1.0 | 18 | — | 1.0 | 5 |
| 2.5 | 1.0 | 100 | 2.5 | 1.0 | 100 |
| — | — | — | 5.0 | — | 87 |
| — | — | — | 5.0 | 1.0 | 100 |

*CC104 is identical with CCcompound104
**CC26 is identical with CCcompound26

⁺Here and elsewhere, 100% inhibition means that no detectable numbers of viable cells remain after treatment.

### Example 8

### Combined Effects of CCcompound104 and CCcompound26 on the Viability of P388/MDR1 Murine Lymphocytic Leukemia Cells

P388/MDR1 cells were seeded either at 10,000 cells per well or 30,000 cells per well in 96-well plates and then incubated for 72 hours in the absence or presence of CCcompound104 and CCcompound26 as indicated in Table 4. When cells were seeded at 10,000 cells per well, 2.5 μg/ml of CCcompound104 alone decreased the number of viable cells by only 52% and addition of 1 μM of CCcompound26 did not significantly improve the inhibitory effect (67% inhibition, Table 4). However, at this lower cell number 5 μg/ml of CCcompound104 alone killed all cells (Table 4). The outcome was similar when cells were seeded at 30,000 cells per well. In this case, 5 μg/ml of CCcompound104 alone decreased the number of viable cells to zero, while the effect of 2.5 μg/ml of CCcompound104 was only slightly enhanced by 1 μM of CCcompound26 (Table 4). Overall, these experiments indicate that MDR1 expressing P388 cells can be effectively killed by 5 μg/ml of CCcompound104 alone, and that addition of CCcompound26 does not dramatically enhance the killing effect of CCcompound104. Since inhibition of MDR1 function alone is not expected to induce cell death, in certain cell lines, such as P388 cells, CCcompound104 must be able to induce cell death by an MDR1-independent mechanism.

TABLE 4
Combined effects of CCcompound104 and CCcompound26 on the
viability of P388/MDR1 cells at lower and higher cell numbers.

| 10,000 cells | | | 30,000 cells | | |
|---|---|---|---|---|---|
| CC104* | CC26** | Inhibition | CC104 | CC26 | Inhibition |
| (μg/ml) | (μM) | (%) | (μg/ml) | (μM) | (%) |
| 2.5 | — | 52 | 2.5 | — | 36 |
| — | 1.0 | 9 | — | 1.0 | 12 |
| 2.5 | 1.0 | 68 | 2.5 | 1.0 | 61 |
| 5.0 | — | 100 | 5.0 | — | 100 |

*CC104 is identical with CCcompound104
**CC26 is identical with CCcompound26

### Example 9

### Combined Effects of CCcompound104 and CCcompound26 on the Viability of Parent L51 78 Mouse T-Cell Lymphoma Cells

Parent L5178 cells were seeded at 10,000 cells per well in 96-well plates and then incubated for 72 hours in the absence or presence of CCcompound104 (1 or 5 μg/ml) and various concentrations of CCcompound26 as indicated in Table 5. CCcompound104 at 1 and 5 μg/ml concentrations decreased the number of viable cells by 40 and 58% respectively. CCcompound26 was also a relatively potent inhibitor alone, and the two compounds together appeared to exert additive rather than synergistic effects (Table 5). This indicates that in the parent cells, MDR1 may not play a significant role in modifying the effects of either compound.

TABLE 5
Combined effects of CCcompound104 and CCcompound26 on the
viability of parent L5178 cells.

| CCcompound104 (μg/ml) | CCcompound26 (μM) | Inhibition (%) |
|---|---|---|
| 0 | 0 | — |
| 1 | 0 | 40 |
| 5 | 0 | 58 |
| 0 | 1.56 | 55 |
| 1 | 1.56 | 72 |
| 5 | 1.56 | 94 |
| 0 | 3.12 | 68 |
| 1 | 3.12 | 97 |
| 5 | 3.12 | 99 |
| 0 | 6.25 | 80 |
| 1 | 6.25 | 100 |
| 5 | 6.25 | 100 |

### Example 10

### Combined Effects of CCcompound104 and CCcompound26 on the Viability of L5178/MDR1 Mouse T-Cell Lymphoma Cells

L5178/MDR1 cells were seeded at 10,000 cells per well in 96-well plates and then incubated for 72 hours in the absence or presence of 5 μg/ml of CCcompound104 and various concentrations of CCcompound26 as indicated in Table 6. CCcompound104 alone did not decrease the number of viable cells. In these cells CCcompound26 alone had much smaller effects than in the parent cells. However, addition of CCcompound104 clearly enhanced the inhibitory effects of CCcompound26 on the viability of MDR1 cells. These data indicate that highly expressed MDR1 effectively reduces the anti-cancer effect of CCcompound26. In addition, the data show that CCcompound104 is suitable to restore the anti-cancer effect of CCcompound26 via inhibiting the activity of MDR1.

TABLE 6
Combined effects of CCcompound104 and CCcompound26 on the
viability of L5178/MDR1 cells.

| CCcompound104 (μg/ml) | CCcompound26 (μM) | Inhibition (%) |
|---|---|---|
| 0 | 0 | — |
| 5 | 0 | 6 |
| 0 | 1.56 | 0 |
| 5 | 1.56 | 31 |
| 0 | 3.12 | 0 |
| 5 | 3.12 | 48 |
| 0 | 6.25 | 10 |
| 5 | 6.25 | 59 |
| 0 | 12.50 | 15 |
| 5 | 12.50 | 77 |
| 0 | 25.00 | 13 |
| 5 | 25.00 | 88 |
| 0 | 100.00 | 56 |
| 5 | 100.00 | 100 |

### Example 11

### Synergistic Effects of CCcompound104 and Ethacrynic Acid (EA) on the Viability of MCF/ADR Cells

MCF/ADR cells were selected by incubating the parent MCF-7 with toxic concentrations (up to 10 μM) of adriamycin (doxorubicin). The selected drug resistant cells express both MDR1 and MRP1 among other molecular alterations as reviewed earlier [Kiss, Z., Tomono, M. and Anderson, W. B. (1994), “Phorbol ester selectively stimulates the phospholipase D-mediated hydrolysis of phosphatidylethanolamine in multidrug-resistant MCF-7 human breast carcinoma cells,” Biochem. J., 302, 649-654]. Treatment of MCF-7/ADR cells with 5 μg/ml of XXcompound104 alone for 72 hours decreased the number of viable cells by 28% (Table 7). Ethacrynic acid (EA) alone, used at 60 μM concentration, decreased cell viability by about 24%, while CCcompound104 and EA in combination had 70% inhibitory effects (Table 7). In this cell line CCcompound104 and EA decreased cell viability synergistically.

In addition to CCcompound104 and EA, the effects of three other CC compounds were also tested. CCcompound26, CCcompound3, and CCcompound19 (all listed in Table 1) even at 50 μM concentration had a 20-24% inhibitory effect on cell viability when employed singly. However, each of these compounds was able to add to the combined inhibitory effects of CCcompound104 and EA (Table 7). Overall, the data suggested that in cells that express both MDR1 and MRP1, a triple combination of CCcompound104, EA and CCcompound26 (or another CC compound listed in Table 1) might be required to exert maximum inhibitory effects on cell viability.

TABLE 7
Combined effects of CCcompound104, ethacrynic acid and
other CC compounds on the viability of MCF-7/ADR cells.

| Treatment | Inhibition (%) |
|---|---|
| CCcompound104, 5 μg/ml | 28.1 |
| EA, 60 μM | 24.3 |
| CCcompound104 + EA | 70.8 |
| CCcompound26, 50 μM | 24.3 |
| CCcompound104 + EA + CCcompound26 | 79.5 |
| CCcompound3, 50 μM | 20.0 |
| CCcompound104 + EA + CCcompound3 | 78.3 |
| CCcompound19, 50 μM | 20.2 |
| CCcompound104 + EA + CCcompound19 | 85.4 |

### Example 12

### Combined Effects of CCcompound104 and CCcompound26 or CCcompound3 on the Viability Peripheral Healthy Lymphocytes

Peripheral lymphocytes are known to be very sensitive to chemotherapy and this sensitivity often limits the dose of chemotherapy that can be employed. In this series of experiments, the combined effects of CCcompound104 alone or in combination with CCcompound26 or CCcompound3 were determined on the viability of peripheral lymphocytes to establish doses that reduce cell viability less than 50%. As in previous experiments, the treatments were performed for 72 hours. In preliminary experiments it was determined that 50 μM CCcompound3 and CCcompound26 decreased viability of healthy lymphocytes by 6 and 100%, respectively. The results of experiments, performed with 4 different lymphocyte preparations derived from 4 healthy volunteers, are shown in Table 8. In none of the cases 5 μg/ml CCcompound104 alone had any detectable (greater than 5%) inhibitory effect on cell viability. However, 5 μM and 1 μM concentrations of CCcompound26 alone decreased viability by 7-59% and 3-13% respectively, while CCcompound3 had no inhibitory effects at either concentration.

In the presence of CCcompound104, addition of 5 μM CCcompound26 decreased viability by 100%, except in one case (46% inhibition), while addition of 1 μM CCcompound26 resulted in less than 50% inhibition. The toxicity profile of co-administered CCcompound104 and 5 μM CCcompound3 was better; the two compounds together reduced viability of normal lymphocytes by 12-27% (Table 8). Based on these data, in subsequent experiments with CLL lymphocytic leukemia cells a fixed concentration of CCcompound104 (5 μg/ml) was used alone or in combination with 1-5 μM concentrations of CCcompound26 or CCcompound3.

TABLE 8
Combined effects of CCcompound 104 as well as
CCcompound26 and CCcompound3 on the viability of healthy lymphocytes.

| | Inhibition (%) | | | |
|---|---|---|---|---|
| Treatments | Volunteer1 | Volunteer2 | Volunteer3 | Volunteer4 |
| CC104, 5 μg/ml* | 0 | 0 | 0 | 3 |
| CC26, 5 μM** | 54 | 7 | 21 | 59 |
| CC104, 5 μg/ml + CC26, 5 μM | 100 | 47 | 100 | 100 |
| CC26, 1 μM | 5 | 3 | 3 | 14 |
| CC104, 5 μg/ml + CC26, 1 μM | 41 | 29 | 47 | 45 |
| CC3, 5 μM*** | — | 0 | 0 | 21 |
| CC104, 5 μg/ml + CC3, 5 μM | — | 12 | 27 | 27 |
| CC3, 1 μM | — | 0 | 0 | 0 |
| CC104, 5 μg/ml + CC3, 1 μM | — | 0 | 0 | 0 |

*CC104 = CCcompound104
**CC26 = CCcompound26
***CC3 = CCcompound3

### Example 13

### Donor CLL Patients

Lymphocytic leukemia cells were collected from 20 patients. The main characteristics of the patients are summarized in Table 9.

TABLE 9
Characteristics of donor CLL patients.

| Patient | Sex | Age | Treatment |
|---|---|---|---|
| 1 | M | 67 | Untreated; in equilibrium |
| 2 | M | 73 | Chlorambucil; Fludarabine |
| 3 | M | 73 | Untreated; in equilibrium |
| 4 | M | 71 | Chlorambucil |
| 5 | M | 63 | Untreated, early stage |
| 6 | M | 73 | Cytoxan, vincristine, steroid |
| 7 | M | 86 | Untreated, stable disease |
| 8 | M | 66 | Fludarabine |
| 9 | F | 67 | Leukeran |
| 10 | M | 73 | Fludarabine |
| 11 | F | 86 | Leukeran |
| 12 | M | 66 | Leukeran |
| 13 | M | 82 | Untreated; in equilibrium |
| 14 | F | 64 | Cytoxan, Fludarabine |
| 15 | M | 77 | Leukeran, Fludarabine |
| 16 | F | 60 | Untreated; in equilibrium |
| 17 | F | 66 | Fludarabine |
| 18 | F | 70 | Untreated; in equilibrium |
| 19 | F | 71 | Untreated; in equilibrium |
| 20 | M | 86 | Untreated; in equilibrium |

### Example 14

### Combined effects of CCcompound104 and CCcompound26 on the Viability of CLL Cells

The aim of these experiments was to determine if a combination of 5 μg/ml of CCcompound104 and 1 μM CCcompound26 is capable of decreasing the viability of CLL cells more than 50% during a 72 hours treatment period. This combination was chosen because in previous experiments this combination decreased viability of healthy lymphocytes by less than 50% (Table 8). Table 10 lists the data obtained with B lymphocytic leukemia cells obtained from 18 CLL patients. In every case, the combined inhibitory effect of CCcompound104 and CCcompound26 was greater than 50% and in 7 cases it reached 100% (Table 10). In 5 cases, CCcompound104 alone had greater than 50% effects on cell viability. Experiments with leukemia cells derived from 2 other patients showing significant drug-resistance is presented in Table 12.

TABLE 10
CCcompound104 and CCcompound26 in combination have greater
effects on the viability of CLL cells than each compound alone.

| | Inhibition (%) | | |
|---|---|---|---|
| | | | CCcompound104 |
| | | | (5 μg/ml) + |
| | CCcompound104 | CCcompound26 | CCcompound26 |
| Patient | (5 μg/ml) | (1 μM) | (1 μM) |
| 1 | 37.5 | 3.5 | 85.7 |
| 2 | 0 | 15.8 | 100 |
| 3 | 62.3 | 66.6 | 100 |
| 4 | 66.6 | 46.2 | 100 |
| 5 | 17.4 | 10.8 | 76.0 |
| 6 | 6.6 | 20.9 | 53.2 |
| 7 | 18.5 | 1.8 | 85.1 |
| 8 | 54.9 | 9.0 | 100 |
| 9 | 55.5 | 9.2 | 90.7 |
| 10 | 44.5 | 40.7 | 96.3 |
| 11 | 55.6 | 48.1 | 88.9 |
| 12 | 39.1 | 30.4 | 95.6 |
| 13 | 35.7 | 42.8 | 100 |
| 14 | 0 | 72.7 | 100 |
| 15 | 22.9 | 1.6 | 95.0 |
| 16 | 0 | 50.0 | 100 |
| 19 | 17.3 | 22.5 | 74.5 |
| 20 | 0 | 0 | 74.4 |

### Example 15

### Combined Effects of CCcompound104 and CCcompound3 on the Viability of CLL Cells

In these experiments, the aim was to determine if a combination of 5 μg/ml of CCcompound104 and 5 μM CCcompound3 is capable of decreasing the viability of CLL cells more than 50% during a 72 hours treatment period. In previous experiments, this combination decreased viability of healthy lymphocytes by less than 30% (Table 8). Table 11 lists the data obtained with lymphocytic leukemia cells obtained from 13 CLL patients. In most cases (10), the combined inhibitory effect of CCcompound104 and CCcompound26 was greater than 50% (Table 11). In case of patients 17, 18 and 20 the combined inhibitory effects of CCcompound104 and CCcompound3 were 0, 19, and 42%, respectively.

TABLE 11
CCcompound104 and CCcompound3 in combination have
greater effects on the viability of responding CLL cells than alone.

| | Inhibition (%) | | |
|---|---|---|---|
| | | | CCcompound104 |
| | | | (5 μg/ml) + |
| | CCcompound104 | CCcompound3 | CCcompound3 |
| Patient | (5 μg/ml) | (5 μM) | (5 μM) |
| 8 | 60.7 | 7.8 | 88.2 |
| 9 | 629 | 11.1 | 81.5 |
| 10 | 50.0 | 32.0 | 94.0 |
| 11 | 62.9 | 33.3 | 92.6 |
| 12 | 47.8 | 26.0 | 95.6 |
| 13 | 30.5 | 12.5 | 80.4 |
| 14 | 0 | 36.3 | 90.9 |
| 15 | 250 | 3.3 | 70.5 |
| 16 | 0 | 50.1 | 100 |
| 17 | 0 | 0 | 0 |
| 18 | 0 | 6 | 18.4 |
| 19 | 20.1 | 9.2 | 57.1 |
| 20 | 0 | 0 | 41.9 |

### Example 16

### Ethacrynic Acid Promotes the Inhibitory Effects of CCcompound104 and CCcompound26 on the Viability of Drug Resistant CLL Cells

CLL cells derived from patients 17 and 18 were highly resistant to the killing actions of CCcompound104 and CCcompound26 (and CCcompound3). Based on the effects of EA in MCF-7/ADR cells, EA (30 μM) was added to the CCcompound104 +CCcompound26-containing treatment regimen. EA alone decreased viability of these resistant CLL cells by 31-38% while in its presence the inhibitory effects of CCcompound104 and CCcompound26 increased from 8-36% to 92-97% (Table 12). Thus, at least in case of these two patients, EA sensitized the cells to the inhibitory actions of CCcompound104 and CCcompound26.

TABLE 12
Ethacrynic acid promotes the combined inhibitory effects of
CCcompound104 and CCcompound26 on the viability
of resistant CLL cells.

| | Inhibition (%) | | |
|---|---|---|---|
| | | CCcompound104 | CCcompound104 (5 μg/ml) + |
| | | (5 μg/ml) + | CCcompound26 |
| | EA | CCcompound26 | (1 μM) + EA |
| Patient | (30 μM) | (1 μM) | (30 μM) |
| 17 | 31.8 | 8.2 | 92.4 |
| 18 | 38.3 | 36.0 | 97.1 |

### Example 17

### Combined Inhibitory Effects of Ethacrynic Acid (EA), CCcompound104 and CCcompound26 on the Viability of Lymphoma Cells Derived from Non-Hodgkin Patients

Lymphoma cells derived from two non-Hodgkin lymphoma patients were treated with EA (15 μM) in the absence or presence of CCcompound104 (5 μg/ml)+CCcompound26 (1 μM) for 72 hours. In both cases EA alone was more effective than the combination of CCcompound104 and CCcompound26 (Table 13). However, the 3 agents together decreased the number of viable cells by 91 and 97%, respectively (Table 13).

TABLE 13
Synergistic inhibitory effects of ethacrynic acid,
CCcompound104 and CCcompound26 on the viability
of lymphoma cells derived from non-Hodgkin patients.

| | Inhibition (%) | | |
|---|---|---|---|
| | | CCcompound104 + | CCcompound104 + |
| | EA | CCcompound26 | CCcompound26 (1 μM) + EA |
| Patient | (15 μM) | (1 μM) | (15 μM) |
| 1* | 36.8 | 14.4 | 91.6 |
| 2** | 42.4 | 23.2 | 97.8 |
| 3*** | 48.2 | 0 | 92.7 |
| 4**** | 53.1 | 0 | 98.4 |

*Patient 1; M, 50 years old, treated with chemotherapy.
**Patient 2; M, 53 years old, treated with chemotherapy.
***Patient 3; M, 80 years old, treated with chemotherapy.
****Patient 4; M, 47 years old, earlier received chemotherapy; presently receives Velcade injection.

### Example 18

### Combined Effects of Epirubicin and CCcompound104 in Parent and MDR1 Expressing Mouse Lymphoma L5178Y cells

Epirubicin, like doxorubicin and other antracyclins, is an MDR1 substrate. The goal of this experiment was to use epirubicin as a model compound to determine if in L5178/MDR1 cells CCcompound104 also can enhance the effects of MDR1 substrate anti-cancer agents other than CCcompound26 and other thioxanthene or thioxanthone CC compounds. The parent and MDR1 expressing cells were seeded at 30,000 cells per well in 96-well microplates and after a 24-h resting period treated with increasing concentrations of epirubicin n in the absence or presence of 5 μg/ml of CCcompound104 for 72 hours. As shown in Table 14, the parent cells were sensitive to the killing actions of epirubicin, and XXcompound104 had little additional effects. On the other hand, MDR1 expressing cells were resistant to epirubicin in the absence of CCcompound104 but became sensitive in the presence of CCcompound104. The results indicate that CCcompound104 should be capable of enhancing the anticancer effects of anticancer drugs in cancers where MDR1 plays a role in the drug-resistance. CCcompound104 is expected to enhance the effects of all anti-cancer drugs that serve as a substrate for the MDR1 protein.

TABLE 14
CCcompound104 promotes the inhibitory effect of
epirubicin on the viability of L5178/MDR1 cells.

| L5178Y | | | L5178/MDR1 | | |
|---|---|---|---|---|---|
| Epirubicin | CC104 | Inhibition | Epirubicin | CC104 | Inhibition |
| (μg/ml) | (5 μg/ml) | (%) | (μg/ml) | (5 μg/ml) | (%) |
| 0 | − | — | 0 | − | — |
| 0 | + | 0 | 0 | + | 0 |
| 0.156 | − | 6.1 | 0.156 | − | 0 |
| 0.156 | + | 3.1 | 0.156 | + | 0 |
| 0.312 | − | 41.5 | 0.312 | − | 0 |
| 0.312 | + | 93.3 | 0.312 | + | 6.0 |
| 0.625 | − | 98.6 | 0.625 | − | 0 |
| 0.625 | + | 99.0 | 0.625 | + | 46.8 |
| 1.250 | − | 100 | 1.250 | − | 0 |
| 1.250 | + | 100 | 1.250 | + | 89.8 |
| 2.500 | − | 100 | 2.500 | − | 12.2 |
| 2.500 | + | 100 | 2.500 | + | 100 |
| 5.000 | − | 100 | 5.000 | − | 32.4 |
| 5.000 | + | 100 | 5.000 | + | 100 |

### Example 19

### Combined Effects of CCcompound104 and CCcompound26 or EA on the Viability of Other Cancer Cell Types

The combined effects of CCcompound104, CCcompound26, and EA were also determined to evaluate the scope of actions of these compounds on the viability of other types of cancer cells, such as cells isolated from solid human tumors. The data are described in the text below.

CaOV-3 is a human ovarian cancer cell line that is often used to study the biology of ovarian cancer. Since CaOV-3 cells highly express MDR1 , it was expected that these cells respond well to this drug combination. Fifty percent decrease in viability was seen in the co-presence of 10 μg/ml of CCcompound104 and 12.5 μM of CCcompound26 with less than 15% inhibition exerted by these compounds when presented to the cells alone. One hundred percent inhibition was observed in the co-presence of 10 μg/ml of CCcompound104 and 25 μM of CCcompound26 with less than 45% inhibition exerted by these compounds when presented to the cells alone. Treatment of CaOV-3 cells with EA (30 μM) alone reduced cell viability by 41%. Importantly, the presence of EA, 5 μg/ml of CCcompound104+1 μM CCcompound26 reduced cell viability by 92%. The data imply that CCcompound104 in combination with CCcompound26 and EA and/or other anti-cancer agents that are substrates of MDR1 may be used to treat ovarian and other MDR1 expressing solid tumors.

The MCF-7 cells were isolated from a human mammary tumor. After combined treatments with 5 μg/ml of CCcompound104 and 1 μM of CCcompound26 for 72 hours, the viability of these cells was decreased by 42%. EA (30 μM) alone decreased cell viability by 32%, while the 3 agents together decreased cell viability about 100% (i.e. practically no viable cells remained after the treatments).

The data obtained with human ovarian and breast cancer cells suggest that certain solid cancers may also be sensitive to the combined killing actions of EA, CCcompound104 and CCcompound26 similar to chronic lymphocytic leukemia and non-Hodgkin lymphoma cells.

1. A method of treating/reducing multiple drug resistance in cancer comprising:
administering to a patient with cancer a composition comprising a xanthene compound alone or in combination with an anti-cancer agent or a glutathione-reducing agent or both.

2. The method of claim 1 wherein the xanthene compound comprises a compound of the formula: 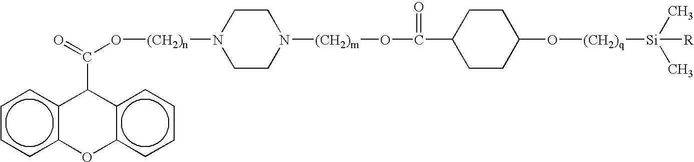
wherein, the molecule contains a heterocyclic hydrophobic moiety such as a xanthene,
wherein, in the general structure n=1-4
wherein, in the general structure m=1-4
wherein, in the general structure q=1-3
wherein, in the general structure R=methyl, ethyl, propyl, butyl, or phenyl
wherein, in the general structure Si may be replaced with N,
wherein, in the general structure the position of the side chain may be ortho or para.

3. The method of claim 2 wherein the heterocyclic moiety is phenoxazine, phenothiazine, thioxanthene, selenoxanthene, or phenazine.

4. The method of claim 1 wherein the xanthene compound is 9H-xanthene-9-carboxylic acid-3 - {4 [2-(4-trimethylsilanyl-methoxy-benzoyloxy)-ethyl]-piperazin- 1 -yl}-propyl ester dihydrochloride, or CCcompound104.

5. The method of claim 1 wherein the xanthene compound inhibits multidrug resistance protein 1.

6. The method of claim 1 wherein the anti-cancer agent comprises a thioxanthene or thioxanthone CCcompound selected from the compounds represented by the formulas:

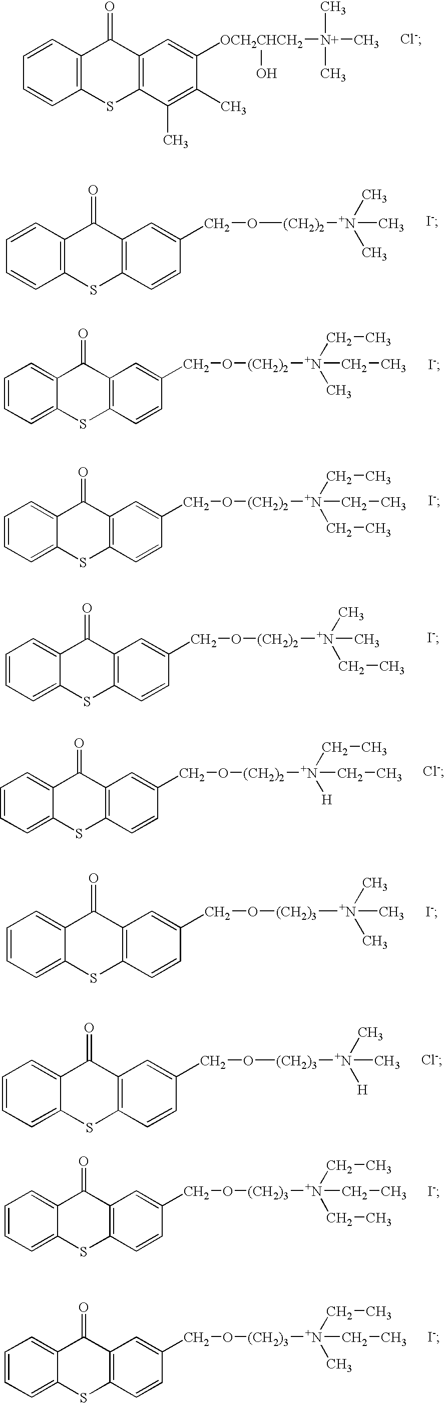

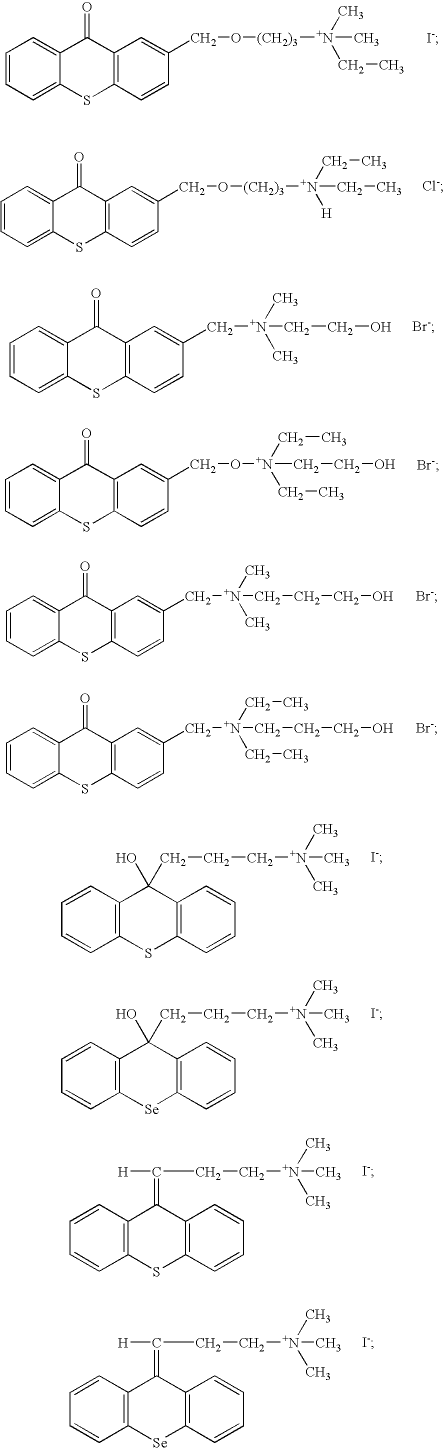

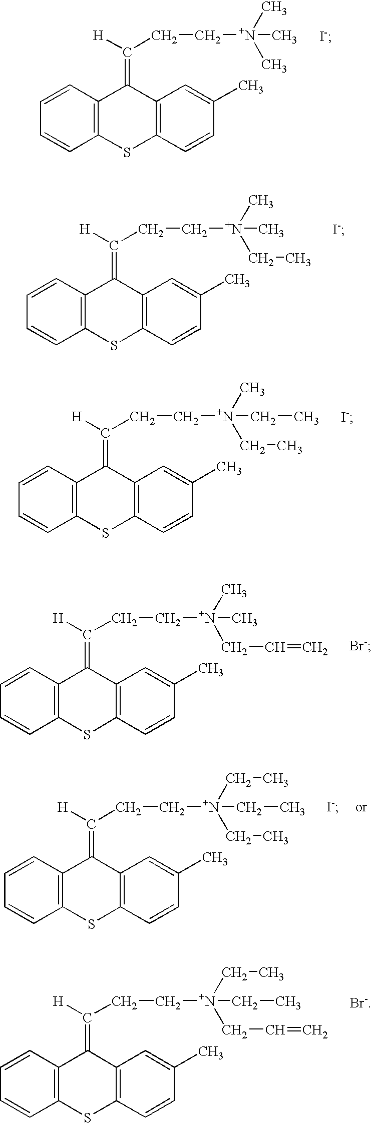

7. The method of claim 6 wherein the CCcompound is CCcompound 26 having the formula:

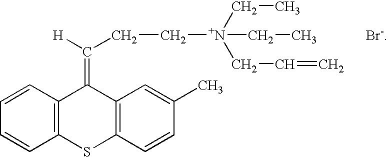

8. The method of claim 1 wherein the glutathione-reducing agent is selected from the group consisting of piriprost, buthionine sulfoximide, 1 -chloro-2,4-dinitrobenzene, and chlorambucil, and ethacrynic acid.

9. (canceled)

10. The method of claim 1 wherein the composition is administered by infusion, subcutaneously by an implanted osmotic minipump, intravenous, intraarterial, subcutaneous, intraperitoneal, intradermal, intratissue, intramuscular, intraportal, intracranial or oral routes.

11. (canceled)

12. The method of claim 1 wherein the composition is administered in the form of a tablet, a gel, or a liquid.

13. The method of claim 4 wherein the dose of orally administered CCcompound4 or an analog is 10-1,000 mg per m² body surface.

14. The method of claim 7 wherein the dose of orally administered CCcompound26 or an analog is 10-1,000mg per m² body surface.

15. The method of claim 4 wherein the dose of injected or infused CCcompound104 or an analog is 5-500 mg per m² body surface.

16. The method of claim 7 wherein the dose of injected or infused CCcompound26 or an analog is 5-500 mg per m² body surface.

17. The method of claim 1 wherein the dose of orally administered glutathione reducing agent is 25-75 mg per m² body surface.

18. The method of claim 1 wherein the dose of injected or infused glutathione reducing agent is 5-25 mg per m² body surface.

19. The method of claim 1 further comprising transplanting autologous or allogeneic hematopoietic stem cells accompanied by the administration of a promoter of survival and proliferation of hematopoietic stem cells such as colony-stimulating factor and granulocyte colony-stimulating factor or a combination of placental alkaline phosphatase and transferrin.

20. (canceled)

21. The method of claim 1 wherein the xanthene compound or its analog is used in combination with agents selected from the group consisting of fludarabine, clofarabine, cladribine, nelarabine, 2-chlorodeoxyadenosine, prednisone, cyclophosphamide, adriamycine, and vincristine, rituximab, alemtuzumab, ritumomab, tiuxetan, tositumomab, etoposid, cisplatin and carboplatin.

22. The method of claim 1 wherein the cancer is chronic lymphocytic leukemia.

23. The method of claim 1 wherein the cancer is non-Hodgkin lymphoma.

24-26. (canceled)

27. A composition for treating or reducing multiple drug resistance in cancer comprising:
a xanthene compound alone or in combination with an anti-cancer agent, a glutathione-reducing agent or both; wherein the xanthene compound comprises a compound of the formula: 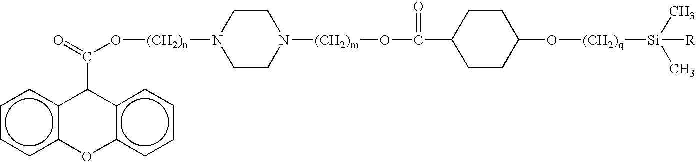
wherein, the molecule contains a heterocyclic hydrophobic moiety such as a xanthene,
wherein, in the general structure n=1-4
wherein, in the general structure m=1-4
wherein, in the general structure q=1-3
wherein, in the general structure R=methyl, ethyl, propyl, butyl, or phenyl
wherein, in the general structure Si may be replaced with N,
wherein, in the general structure the position of the side chain may be ortho or para.

28. (canceled)

29. The composition of claim 27 wherein the xanthene compound is 9H-xanthene-9-carboxylic acid-3 - {4 [2-(4-trimethylsilanyl-methoxy-benzoyloxy)-ethyl]-piperazin-1-yl}-propyl ester dihydrochloride, or CCcompound104.

30. (canceled)

31. The composition of claim 27 wherein the anti-cancer agent comprises a thioxanthene or thioxanthone CCcompound selected from the compounds represented by the formulas:

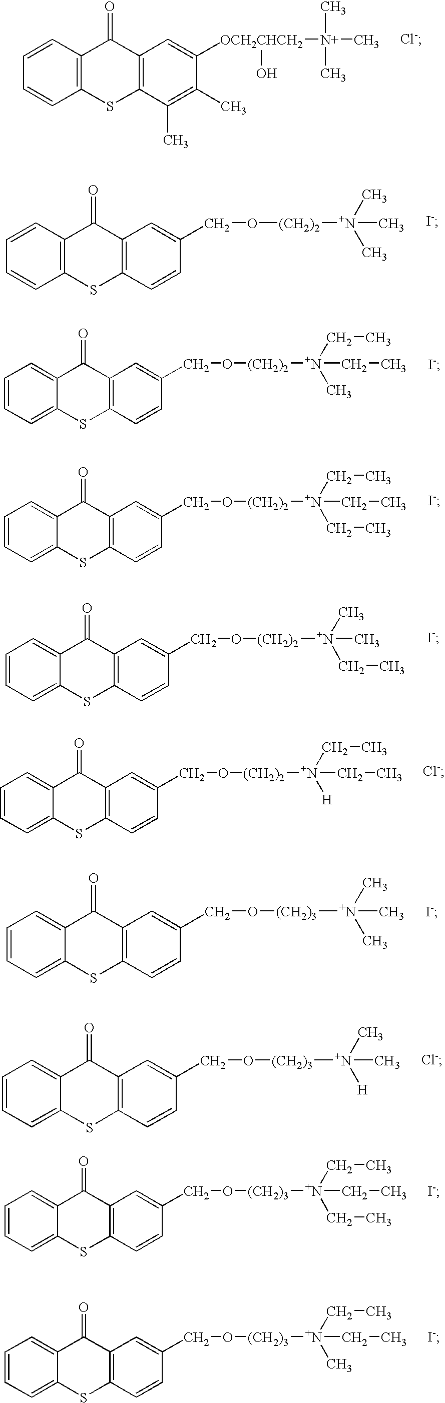

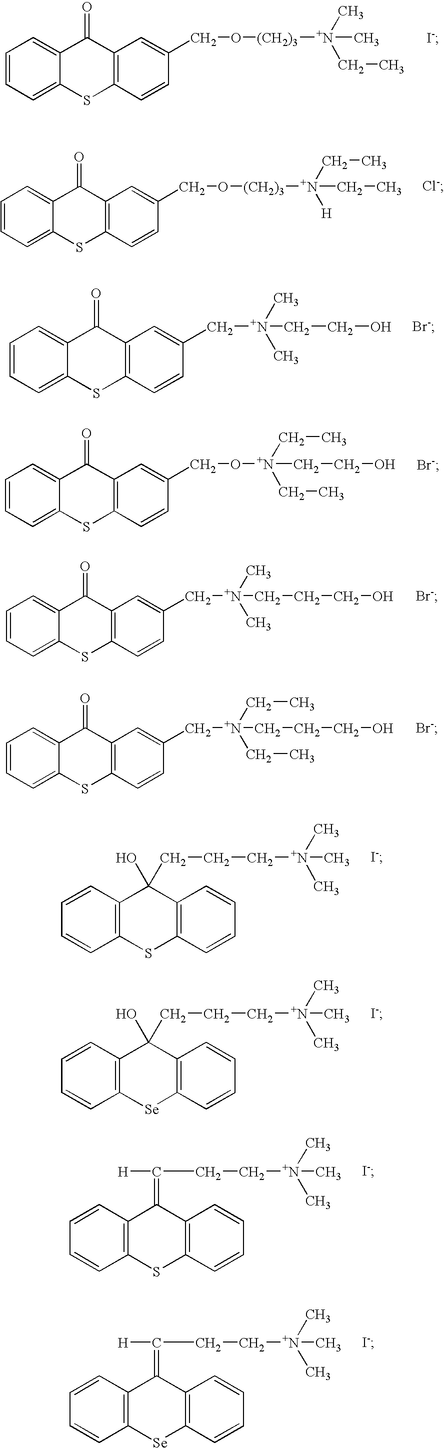

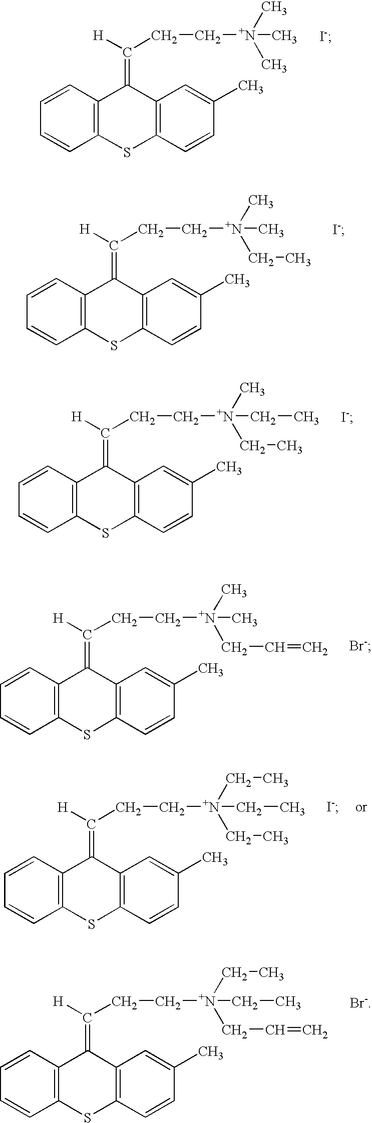

32. (canceled)

33. The composition of claim 27 wherein the glutathione-reducing agent is piriprost, buthionine sulfoximide, 1 -chloro-2,4-dinitrobenzene, chlorambucil or ethacrynic acid.

34-35. (canceled)

36. The compound of claim 27 wherein the heterocyclic moiety is phenoxazine, phenothiazine, thioxanthene, selenoxanthene, or phenazine.

37-38. (canceled)
